(19) United States
(12) Patent Application Publication
(10) Pub. No.: US 2023/0321291 A1
(43) Pub. Date: Oct. 12, 2023

(54) SYSTEM HAVING A RADIATION SOURCE, A SENSOR AND A MOBILE TERMINAL FOR DETECTING SURFACE STRUCTURES AND ANOMALIES

(71) Applicant: AR-CHECK GMBH & CO. KG, Frankfurt am Main (DE)

(72) Inventor: Martin CUDZILO, Frankfurt am Main (DE)

(73) Assignee: AR-CHECK GMBH & CO. KG, Frankfurt am Main (DE)

(21) Appl. No.: 18/025,127

(22) PCT Filed: Sep. 8, 2021
(86) PCT No.: PCT/DE2021/100740

§ 371 Date: Mar. 7, 2023

(30) Foreign Application Priority Data
Sep. 8, 2020 (DE) 10 2020 123 423.6

(51) Int. Cl. A61L 2/10; A61L 2/24; A61L 2/28; G06F 1/16; G02B 27/01

(52) U.S. Cl. CPC A61L 2/10; A61L 2/24; A61L 2/28; G06F 1/163; G02B 27/0172; A61L 2202/14; A61L 2202/11; A61L 2202/16; G02B 2027/0178

(57) ABSTRACT

A system, having at least one radiation source and at least one mobile terminal, which has at least one optical sensor and a data-processing device, wherein the at least one optical sensor of the at least one mobile terminal is designed and configured to receive reflected radiation of the radiation source, so that surface structures, more particularly fouling, are detected from the received radiation of the radiation source by means of the data-processing device and, in particular, are stored in the data-processing device.

The present invention relates to a system having at least one radiation source and having at least one mobile terminal having at least one optical sensor for detecting radiation of the radiation source reflected by the surroundings, and having at least one data processing device in order to be able to detect surface structures, in particular contaminants.

In order to ensure sufficient hygiene and cleanliness, particularly in buildings, surfaces are cleaned at regular intervals. In this case, surfaces may be formed from various materials, such as wood, stone, glass and/or plastic, and may be situated outdoors, such as e.g. benches, or in interiors of buildings, such as e.g. floors, pieces of furniture, wall sections, and the like. Moreover, surfaces may comprise sections of or entire exterior facades, such as wall panels, windows, and the like.

Furthermore, it is advantageous if surface constitutions and structures can generally be detected, for example for the purpose of material testing, quality inspection, etc.

Both during cleaning and during quality inspection, it is advantageous if the structure of a surface can be detected by way of metrology and conditioned by means of data processing devices.

In this case, surfaces are cleaned mainly when the fewest possible persons are adversely affected by the cleaning process. In addition, for economic reasons, the cleaning duration will if possible be kept as short as possible in order to save work time and thus costs. The objective is always to carry out the most efficient cleaning possible—i.e. with the best cleaning result with the least possible use of material and work time.

Concurrently, customers have been demanding increased convenience and very recently, in particular, hygiene requirements have also increased in many areas, primarily in connection with preventing pandemics such as may be caused by influenza viruses, for example. Special hygiene measures are necessary particularly during pandemics, moreover, in order to stop them spreading.

In order to enable cleaning which is as efficient as possible and which at the same time satisfies the increased convenience and hygiene requirements, technical aids which assist a user during cleaning and aids which inspect the cleaning result are desirable.

Furthermore, it would be advantageous to be able to have recourse to a system which supports or ensures disinfection within the scope of the hygiene requirements.

Comparable requirements arise during the analysis of surfaces with regard to possible material testing. These tests too—in as much as they are not carried out in the course of or together with production—are intended to be carried out as efficiently as possible and generally take place outside normal operation in the context of maintenance, etc.

For ensuring hygiene in buildings, in the prior art it is known in principle to use apparatuses which have an optical detection device, which generate electromagnetic radiation for irradiating a surface to be cleaned, and which make visible contaminants that are invisible to humans under visible light. There is usually recourse to UV light for this purpose.

Such an inspection apparatus can ensure that even those contaminants which cannot be seen by the cleaning personnel without technical aids can be made visible.

Such apparatus are known in the prior art and, in the simplest case, consist of a UV lamp, which is optionally used together with polarized spectacles. Such systems are known from physicists for securing evidence, for example, in which usually use is also made of an aid, such as luminol, which interacts with specific substances—in the case of luminol with blood or more precisely the hemoglobin in blood.

For the direct use of UV light optionally with a polarization filter without optical image processing, partial darkening is usually necessary as well in order to enable sufficient visibility of the reflected UV light from the surfaces.

Moreover, the prior art discloses systems which capture UV light emitted by a radiation source by means of a camera and make it visible to a user on a monitor. These systems are used in clean rooms, for example, in which any impurities at all must be avoided. These clean rooms are closed areas which can be efficiently equipped with stationary systems.

Both variants mentioned allow, firstly, detection of contaminants possibly still present during the cleaning process and the cleaning of dirty surfaces and, secondly, also inspection regarding immaculate cleaning.

As a result, the cleaning quality is improved and the required hygiene is thus ensured by virtue of the fact that not only the “visible” dirt but also contaminants that are not visible to the human eye without aids can be detected and removed.

As a result of contaminants that are difficult to discern or are indiscernible being made visible, a time saving is made possible, moreover, both for the cleaning process itself and for the inspection since the inspection device can be accessed at any time.

A comparable situation also applies to material testing. By emitting radiations of specific wavelengths, it is possible to make visible cracks and surface structures which would be indiscernible to the user without technical aids.

What is disadvantageous about the described solutions and comparable solutions is that either they take place virtually exclusively manually—the user aligns his/her UV lamp or radiation source and sees the contaminated surface—or they are installed in a stationary or fixed manner.

This may in both cases be expedient for selected applications, but for many applications is impracticable or can only be realized with very great outlay. Furthermore, this usually does not directly help the cleaning personnel who are cleaning, but rather serves to provide a first overview and subsequent inspection before and/or after cleaning, for which the cleaning process has to be interrupted in each case.

There is therefore no direct real-time feedback which enables cleaning success to be monitored by the user during cleaning.

The object of the invention, then, is to overcome the disadvantages of the prior art, and in particular to provide an apparatus and a method which not only improves cleaning but also makes it more efficient economically, in particular real-time feedback for monitoring the cleaning result being provided.

This object is achieved by means of a system comprising at least one radiation source and at least one mobile terminal having at least one optical sensor and having a data processing device, wherein the at least one optical sensor of the at least one mobile terminal is designed and configured to receive reflected radiation of the radiation source, such that surface structures, in particular contaminants, are detected from the received radiation of the radiation source by means of the data processing device and are stored in particular in the data processing device.

According to the invention, it has proved to be particularly advantageous to provide a system which comprises a mobile terminal having an optical sensor and also a radiation source. In this case, the mobile terminal can detect the radiation emitted by the radiation source by means of the at least one optical sensor and can evaluate the sensor data being received in a data processing device in order to determine surface structures and make them visible to a user. This concerns in particular contaminants on surfaces, but can also relate to material damage, cracks, etc. in and/or on the surfaces.

An apparatus according to the invention makes it possible in particular to increase the cleaning quality of cleaning with higher economic viability at the same time, since on the mobile terminal the user not only initially obtains an overview, but can track the cleaning result during cleaning—on request in real time during the entire cleaning process. In this case, in particular, contaminants that are poorly visible or not visible to the user without technical aids become visible to the user.

The same applies, mutatis mutandis, to a quality inspection of surfaces, which can be carried out in real time by means of the mobile terminal and gives the user direct feedback about the surface structure.

In this case, the term cleaning quality can be subdivided into the areas of cleanliness and hygiene. The term “cleanliness” encompasses in particular visual cleanliness or else the aspect of odor nuisance. The term “hygiene” encompasses in particular a possible health hazard for the user.

A user shall generally be understood to mean a person who in the broader sense interacts with the system according to the invention. This may be a cleaner, an inspector who inspects the cleaning result, a supervisor who supervises employees, a client that uses the system according to the invention for inspection purposes or for recommissioning work, and further persons who carry out the aforementioned or further tasks directly or indirectly in connection with the system according to the invention.

Surfaces to be cleaned can be for example entire floor surfaces, walkways, floor edge regions, tabletops, table bases, front panels of closets, top edges of closets, WCs, hand washbasins and the like, or portions thereof, the present enumeration being clearly by way of example and not exhaustive.

In this case, a system according to the invention can also be used for quality control inspections and “making visible” to the human eye properties of surfaces that are difficult to discern or indiscernible without technical aids.

In this case, the system according to the invention combines the advantages of (exclusively manual) variable analyzing of surface structures, in particular of contaminants of surfaces, with the otherwise exclusively stationary recording of same by means of cameras, in particular the data processing device according to the invention additionally being used.

In contrast to the prior art, the system according to the invention thus has the advantage that the user himself/herself can always set the desired recording direction of the camera, whereas the recording angles are predefined in the case of devices in accordance with the prior art. The system according to the invention is distinctly more flexible here than the devices and systems known from the prior art, and in this case the respective recording angles or recordings can be directly stored and processed further by the data processing device.

In this case, the data processing device of the mobile terminal serves for identifying properties of surfaces for the purpose of ascertaining the structures thereof, in particular for identifying contaminants, on the basis of the raw data detected by the at least one optical sensor.

In this case, provision can be made for this identifying to take place completely in the data processing device in the mobile terminal. However, alternatively, provision can also be made for the data processing device to forward the detected raw data and/or partly processed data to a further data processing device at a server and for further processing of the data to take place there. In this case, the further data processing device can communicate the processed data back again to the data processing device in the mobile terminal in order that the latter data processing device controls a display device to display a representation of the contaminant to the user.

In the simplest case here the representation of the data can be identical with the recorded images. However, according to the invention, provision can likewise be made for using image processing algorithms which condition the data being received and display the data that have been conditioned in an improved manner to the user.

Such algorithms for identifying patterns and structures are known in the prior art and may be chosen by a person skilled in the art according to the respective requirements.

In this case, the system according to the invention makes possible for a user, by means of the at least one radiation source comprised, for example a UV light source, which can be embodied such that it is freely positionable in space, and the mobile terminal having the at least one optical sensor, for example in the form of a camera, and the data processing device, a freely selectable view of surfaces in space, wherein surface structures, in particular impurities, that are not directly visible to the human eye become directly visible and/or are optically conditioned by means of the data processing device.

In this case, it can be provided in particular that the radiation source is embodied as a mobile radiation source and comprises a rechargeable battery and/or is connected to a rechargeable battery of the mobile terminal. According to the invention, it can be provided that the at least one radiation source is positioned independently of the mobile terminal in space. In this case, it has proved to be particularly advantageous if the at least one radiation source is likewise embodied in mobile fashion and in particular comprises a dedicated power supply by means of a rechargeable battery, such that it is freely positionable in space.

Alternatively or additionally, it can be advantageous for the at least one radiation source to form a unit with the mobile terminal and also to be connected or connectable to the energy supply thereof.

A multiplicity of radiation sources are conceivable. UV light sources have proved to be particularly advantageous as radiation sources within the meaning of the invention. However, for embodiments of the invention, also radiation sources which emit light in the blue light spectrum, for example, or radiation sources which emit infrared light are likewise advantageous in order to detect surface structures.

In accordance with the prior art, UV light sources have been embodied as gas discharge lamps, for example. These lamps required relatively high voltages and had a relatively high power consumption. Alternatively, traditional illuminants were known, too, which as “black light lamps” comprised polarization filters, such as e.g. incandescent bulbs or fluorescent tubes. However, very energy-saving LEDs— light emitting diodes—or OLEDs have been known for some time, which can be supplied with energy by way of the rechargeable battery of a mobile terminal without any problems.

LEDs and OLEDs are likewise known which emit blue light, infrared light, or light or radiation of other wavelengths, and which can be advantageous for embodiments of the invention. In particular, provision can also be made for the radiation source to be embodied in the form of a laser.

In this case, it can be provided in particular that the mobile terminal comprises a display device, wherein the display device is designed and configured to reproduce a representation of the recordings obtained by the optical sensor and/or of the surface structures identified from the reflected radiation by means of the data processing device, in particular the identified contaminants. If the display device is directly connected to the mobile terminal, this makes possible a particularly compact design of the system and the mobile use thereof. In a different case, it can be provided that the display device is spatially separated from the terminal and an identification of surface structures, in particular of the contaminants, from a distance is thus possible. In this regard, by way of example, a consultation between the cleaner and the supervisor, i.e. two optional users of the system according to the invention, can take place without the supervisor having to be on site in order to assess contaminants and/or the cleaning carried out. In this case, it is particularly advantageous if the cleaner has at least one display device and the supervisor has at least one further display device.

In this case, in particular, in accordance with one embodiment, it can also be provided that a supervisor is not necessary, rather that algorithms stored in the data processing device are used to perform an automated inspection of work carried out and the result thereof, wherein the user then obtains feedback regarding the work carried out, in an automated manner by means of the algorithm.

Furthermore, it has proved to be advantageous if the identified surface structures, in particular the identified contaminants, are highlighted and/or marked by the display device. This can be done by coloring the surface structure, in particular the contaminant, and/or marking the surface structure, in particular the contaminant, with a circle or arrow, the present enumeration clearly being by way of example and not exhaustive.

In this case, it can be advantageous in particular if the display device reproduces the cleaning process. This can be done, by way of example, by the coloring of the contaminant disappearing in the course of cleaning or by the locations that have already been cleaned being colored differently. Furthermore, provision can be made for the mobile terminal to be smartglasses, a smart helmet, a smartphone, a tablet, a notebook or a netbook.

These types of mobile terminal are known to a person skilled in the art in various embodiments with and without a camera and/or a data processing device and enable mobile and stationary use of the system, in particular the smartglasses being suitable for being used by the cleaning personnel during the cleaning process without obstructing the work process.

In one embodiment, the lenses of the smartglasses and/or of the smart helmet comprise a filter, embodied to filter at least 20%, preferably at least 50%, particularly preferably 100%, of light having a wavelength of below 100 nm and having a wavelength of above 490 nm, in particular of above 380 nm.

It can be provided that specific surface structures or contaminants can be seen particularly well in the above wavelength spectrum, in particular under blue light or UV light, including preferably under black light. If other wavelengths of radiation are filtered, disturbances for instance as a result of reflections in the visible light range can be avoided, and the user obtains clearer highlighting of the surface structures by means of the smartglasses or the smart helmet.

Alternatively, it can be provided that the lenses of the smartglasses and/or of the smart helmet comprise a filter, embodied to filter at least 20%, preferably at least 50%, particularly preferably 100%, of light having a wavelength in the range of 100 nm to 380 nm. UV light is known to be able to damage the human eye.

For this reason, it can be advantageous if the lenses of the smartglasses or of the smart helmet are designed and configured to protect the eye in particular against this radiation, wherein the display device integrated into the lenses of the smartglasses or of the smart helmet enables the reproduction of the contaminants detected by means of the camera.

Furthermore, according to the invention, it can also be provided that when a smartphone, tablet, notebook or netbook is used as mobile terminal, the user wears UV protective goggles that correspondingly filter UV light.

Moreover, according to the invention, it has proved to be advantageous that the at least one radiation source in particular consists of at least one light emitting diode or the latter is comprised by the radiation source.

With the use of radiation sources, in particular UV light sources, it is often necessary to conduct an appraisal weighing up the required radiation energy to be emitted and the peak range and also the possibilities for the power supply and desired or required compactness of the UV light sources. Since an at least partly compact design has proved to be advantageous according to the invention, it is advantageous for UV light sources used to be light emitting diodes, at least in part, which are characterized by spatial compactness and can be supplied with energy by way of the rechargeable battery of a mobile terminal without any problems.

Furthermore, according to the invention, it can also be provided that the light emitting diode is supplemented by further UV light sources, in particular light emitting diodes, in particular in order to achieve complete illumination of the area that is detectable by the at least one optical sensor, for example embodied in the form of a camera, of the mobile terminal.

Furthermore, it can be advantageous that the at least one radiation source is arranged on the mobile terminal, wherein in particular the at least one radiation source and the at least one optical sensor are movable or are moved equidirectionally

The compact design of the at least one radiation source and the possibility of covering the energy demand by way of rechargeable batteries make possible the arrangement according to the invention of the at least one radiation source, in particular of LEDs or OLEDs, in particular those which emit UV light, on the mobile terminal. This arrangement makes it possible to install the at least one radiation source equidirectionally with the at least one optical sensor and thus to optimally illuminate the detection field thereof and in addition to move these equidirectionally.

With the use of smartglasses or a smart helmet as mobile terminal, said smartglasses or smart helmet can be worn by the user without any problems, for example by the cleaner in the case of cleaning, during the cleaning process without hindrance and restriction, and at the same time the surface structure, for example the areas that are to be cleaned or have already been cleaned, is displayed to the user.

According to the invention, it can be advantageous that the data processing device conditions the image captured by the at least one sensor by means of imaging methods and identifies specific elements on a surface detected by way of the reflected radiation of the radiation source, in particular contaminants, viruses, fungal spores and the like.

Conditioning can be effected, by way of example, by pattern recognition techniques known to a person skilled in the art. In particular, the identification of the reflected UV light is appropriate here, this light being a good measure of the degree of soiling. Moreover, differences in the wavelength of the incident and received light can provide information about the types of contaminant. If the camera is designed and configured to detect emission phenomena such as fluorescence or phosphorescence, in particular, further information about the contaminants can be obtained.

In particular, the UV light source according to the invention, i.e. a light source which generates ultraviolet light and which preferably has a peak of the radiation emission in the wavelength range of between 100 nm and 380 nm, and thus irradiates the area to be cleaned or to be inspected, makes it possible to identify even contaminants which at first glance are difficult to discern or are not discernible at all by the human eye under natural light or normal ambient light in the interior.

In this case, UV light is subdivided into UV-A with a wavelength in the range of 315 to 380 nm, UV-B with a wavelength in the range of 280 to 315 nm, and UV-C with a wavelength in the range of 100 to 315 nm. A peak should be understood to mean that at least 50% of the emitted radiation energy of the radiation source, in particular at least 75% of the energy, lies in the specified wavelength range. A UV light source thus emits at least 50% of its energy in the UV light spectrum.

Furthermore, it has proved to be advantageous if the data processing device generates a graphical representation of the identified elements and displays it to the user on the display device.

Recognized surface structures, such as, for example, cracks, elevations, depressions, etc., and identified contaminants can be highlighted and/or marked by the display device. This can be done by coloring the recognized surface structures, in particular the contaminant, and/or marking the surface structures, in particular the contaminant, with a circle or arrow, the present enumeration clearly being by way of example and not exhaustive. This is made possible by the images captured by the at least one optical sensor being conditioned by a data processing device by means of imaging methods, the data processing device being operatively connected to the display device.

In particular, this method also makes it possible to specially highlight contaminants such as, by way of example, viruses and/or fungal spores.

According to the invention, it is additionally advantageous if the graphical representation is displayed to the user in a superimposed manner, in particular partly transparently, on the display device of smartglasses and/or a smart helmet.

With these types of presentation, the cleaning process can be tracked and locations that have not been cleaned can be identified. This enables deficiencies to be rectified properly.

Furthermore, it can prove to be advantageous that at least one further radiation source is provided in addition to the at least one radiation source on the mobile terminal and also emits radiation. In this case, provision can be made for the at least one further radiation source, in particular in the form of a UV light source, to be used separately from the user, for example in the form of a portable lamp, wherein said at least one further radiation source serves either for illuminating the space for subsequent detection by means of the at least one optical sensor for example for disinfecting surfaces by means of UV light.

Furthermore, it can be advantageous that at least one further optical sensor is provided in addition to the optical sensor on the mobile terminal, the data of which are evaluated by the data processing device in parallel or alternately with the data of the optical sensor on the mobile terminal. In order to detect surfaces as comprehensively as possible and in particular to make possible a good cleaning result, it is expedient for the relevant surfaces, in particular the areas to be cleaned, to be illuminated from different angles and from different locations and to be detected by way of imaging in order to detect or ascertain as many and/or varied contaminants as possible.

In this case, the information acquired can also be used in particular for generating a virtual reality and/or augmented reality environment. Moreover, a redundant design of the camera system affords increased reliability.

According to the invention, it can additionally be advantageous that at least one third sensor is comprised in addition to the optical sensor on the mobile terminal in order to detect radiation reflected by the radiation source(s), wherein the data processing is designed and configured to process the data of the at least one third sensor exclusively or in addition to the data of the at least one first sensor or of the at least one further sensor, in order to detect contaminants.

Depending on the type of surface or surface structure to be detected, in particular the type of a possible impurity, different sensors are suitable for detecting impurities. By way of example, it may be the case that viruses and fungi can be detected in particular with the aid of UV light, but other contaminants, such as a coffee spot, by way of example, are not detected. Moreover, imaging methods are not able to detect odors and the source thereof; that necessitates other sensors such as mass spectrometers or chromatographic methods.

In accordance with a further exemplary embodiment, the system has a, more particularly optical, sensor for detecting electromagnetic radiation in a defined wavelength range that emanates from the area to be cleaned, depending on the contaminant. The sensor is additionally embodied to generate a corresponding (measurement) signal.

It can also be preferred for the at least one first sensor and/or the at least one further sensor to be a camera.

As already explained beforehand, a camera is an embodiment of an optical sensor that is particularly preferred according to the invention.

Furthermore, it can be preferred for the at least one radiation source and/or the at least one further radiation source to emit radiation with a wavelength in a range of 10 nm to 490 nm, in particular in a range of 280 nm to 380 nm, to the extent of, in particular, at least 80% of the emitted radiation energy relative to the total radiation energy of the respective radiation source.

Finally, it can also be provided that the radiation source is designed and configured to disinfect a surface.

In accordance with one embodiment of the present invention, it can be preferred for the at least one radiation source to emit radiation that serves for disinfecting surfaces. In this case, in particular, UV radiation and/or radiation with a wavelength range in the blue light spectrum can be used.

In this case, UV radiation has the advantage that it achieves a good disinfecting effect with a high energy density. However, UV light can be harmful to the human eye and may possibly have a negative effect on the lifetime of specific materials such as plastics. In comparison with UV light, blue light has only a weak disinfecting effect, but in return it is generally harmless to humans and materials.

Moreover, the invention provides a mobile terminal comprising a system according to the invention.

In this case, according to the invention, it can be provided in particular that the mobile terminal comprises at least the one optical sensor, the display device, the at least one radiation source and the data processing device.

Moreover, the invention provides a method for operating a system according to the invention or a mobile terminal according to the invention, comprising the following steps, in particular in this order:
a) activating the radiation source;
b) detecting the radiation of the radiation source reflected by the surroundings by means of the at least one sensor and/or the further sensor; and
c) evaluating the detected sensor data in the data processing device and generating a virtual representation of surface structures, in particular of contaminants.

In this case, it can be advantageous that the method according to the invention furthermore comprises the following step:
d) displaying the virtual representation of the surface structure on a display device, in particular on smartglasses or a smart helmet, wherein the displaying is displayed in particular in the form of augmented reality as additional information for a user.

Moreover, the method can furthermore comprise the following step:
f) documenting and recording the changes in the space detected by the at least one sensor or the further sensor and/or the work carried out by the user.

Furthermore, the method according to the invention can comprise the following step:
g) disinfecting an object and/or a spatial region by means of one of the at least one radiation source or the further radiation source of the system or of the mobile terminal.

Finally, the invention provides a use of a system according to the invention or of a mobile terminal according to the invention for assistance and optional documentation of surface structures, in particular of contaminants, cleaning processes, repairs and/or quality inspections.

The system according to the invention and the mobile terminal according to the invention are also suitable for other fields of use, e.g. in industry for the purpose of analysis, material testing or detection of damage, anomalies, etc. (with contrast media being employed), verification of documents, crime scene inspection, etc.

There are many advantageous applications in which features or contaminants that are visible to the human eye only with difficulty if at all can be made detectable in real time.

Further advantages and features of the invention will become apparent from the following description which explains exemplary embodiments of the invention by way of example with reference to a schematic drawing, without the invention being restricted thereby.

In the drawing:

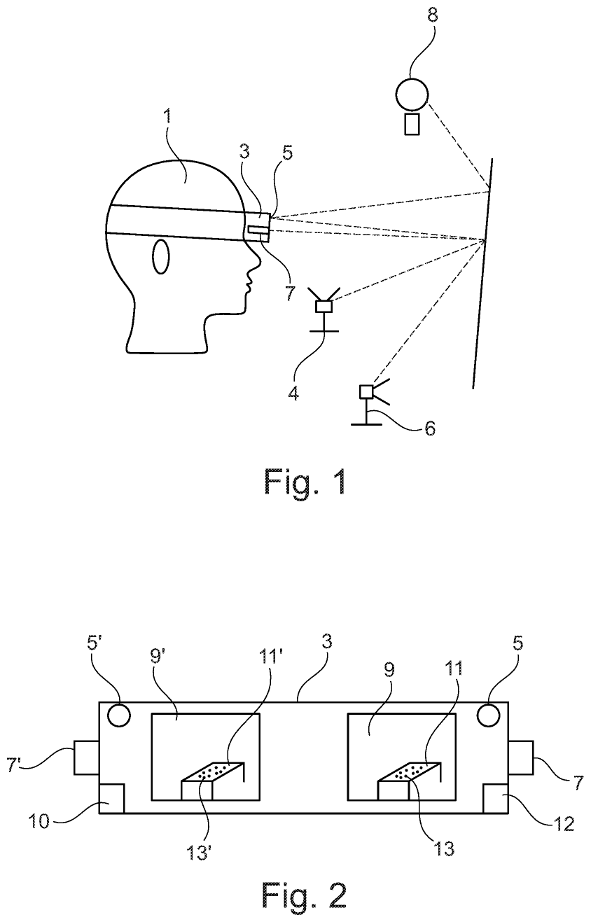

FIG. 1: shows a schematic side view of one exemplary embodiment of a system according to the invention;

FIG. 2: shows a schematic front view of the embodiment from FIG. 1 of the mobile terminal;

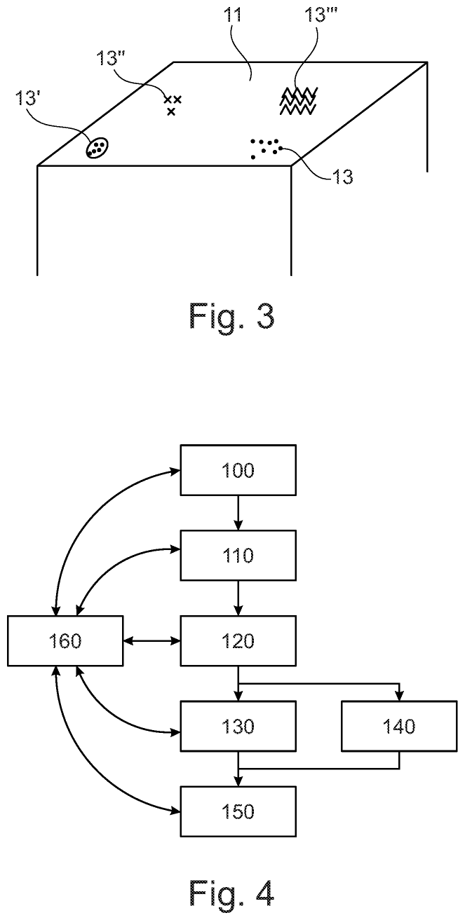

FIG. 3: shows a schematic perspective view of a virtual representation of an object to be cleaned; and

FIG. 4: shows an exemplary sequence of a method according to the invention.

FIG. 1 shows by way of example one embodiment of a terminal 3 according to the invention. A cleaning person 1 as an example of a user here is wearing the mobile terminal 3 in the embodiment of smartglasses comprising a camera 5 as an example of an optical sensor according to the invention and a UV light source 7 as an example of a radiation source, and furthermore comprising a further additional UV light source 8 as a further radiation source, a further additional sensor 4 and a further additional camera 6.

As is readily evident in FIG. 1, the additional UV light source 8 can be positioned as desired in space and, by way of the additionally emitted light, can generate further reflections that can be detected by the camera 5 or the additional camera 6.

FIG. 2 shows by way of example the mobile terminal 3 in the embodiment of smartglasses comprising the cameras 5, 5′ and UV light sources 7, 7′, and also a data processing device 10 and a rechargeable battery 12, with the display devices 9, 9′ and also an area 11, 11′ to be cleaned and the impurity 13, 13′ furthermore being shown.

Various work modes are conceivable here. The smartglasses can display a “virtual” reality as smartglasses, but they can also preferably be embodied as augmented reality (AR) glasses. In this case, the reality seen by the user is superimposed with additional information, as described above.

In this case, provision can be made in particular for the camera 5 and the additional sensor 4 to detect data in real time. From these data, the data processing device 10 generates a presentation of the contamination, as shown by way of example in FIG. 3 below, and projects this and optionally additional information for the cleaner into the smartglasses 3, embodied as AR glasses, into the field of view during cleaning. In addition, these generated data can be used for documentation and/or for optimization of the cleaning or work sequences, etc.

During the entire process, the smartglasses or the augmented reality glasses can inspect, document and evaluate the cleaning process and the result.

If a system according to the invention is used in a hospital, for example, then over and above the cleaning personnel the system has great benefits e.g. for nursing staff, whose occupation causes them to be in (skin) contact with humans, but also for use in hygiene inspections, etc.

FIG. 3 shows by way of example a dirty area or area to be cleaned having the impurities 13, 13′, 13″ and 13′″. The impurity 13 is an impurity that is displayed directly by way of reflected UV light that is visible to the user in the smartglasses 3.

The impurity 13′ is an impurity that is marked by a circle. Of course, other kinds of highlighting such as arrows, indications, etc., are also conceivable, where the kind of highlighting can be appropriately defined by a person skilled in the art depending on the application.

The impurity 13″ is a virtual representation of an impurity that is marked by crosses. It goes without saying that such “virtual” impurities can also be highlighted using graphical elements in accordance with 13′.

Primarily if the smartglasses 3 comprise a UV filter in order to protect the user's eyes, said filter completely or partly filtering UV light, exclusively virtually calculated representations of impurities are displayed to the user.

In this case, but also in accordance with the further embodiments of the present invention, impurities can be displayed by way of chosen virtual representations thereof.

A further alternative form of presentation of an impurity is impurity 13′″. In this case, the individual impurity is not displayed, rather a calculated severely dirty region is highlighted as a complete region graphically on a surface to be cleaned.

It is evident here to a person skilled in the art that the various forms of presentation of impurities can be generated by the data processing device 10 and displayed on the display device 9, 9′ individually and in any possible combination.

FIG. 4 schematically shows the method steps according to the invention for operating a system or a mobile terminal, wherein step 100 represents activating the UV light source, step 110 represents detecting the UV light of the UV light source reflected from the surroundings by means of the camera or a sensor; and step 120 represents evaluating the detected camera and/or sensor data in the data processing device and generating a virtual representation of contaminants, step 130 represents displaying the virtual representation of the contaminants on a display device, in particular on smartglasses, wherein the displaying is displayed in particular in the form of augmented reality as additional information for a user, step 150 represents documenting and recording the changes in the space detected by the camera or the sensor and/or the work carried out by the user, wherein step 140 denotes displaying work instructions and/or information in parallel with or as an alternative to displaying the virtual representation of contaminants, and step 160 denotes disinfecting an object and/or a spatial region by means of one of the UV light sources of the system or of the mobile terminal.

The features of the invention disclosed in the above description, the claims and the drawings may be essential to the realization of the invention in its various embodiments both individually and in any desired combination.

1. A system comprising at least one radiation source and at least one mobile terminal having at least one optical sensor and having a data processing device, wherein the at least one optical sensor of the at least one mobile terminal is designed and configured to receive reflected radiation of the radiation source, such that surface structures, in particular contaminants, are detected from the received radiation of the radiation source by means of the data processing device and are stored in particular in the data processing device.

2. The system as claimed in claim 1, wherein
the radiation source is embodied as a mobile radiation source and comprises a rechargeable battery and/or is connected to a rechargeable battery of the mobile terminal.

3. The system as claimed in claim 1, wherein
the mobile terminal comprises a display device, wherein the display device is designed and configured to reproduce a representation of the recordings obtained by the optical sensor and/or of the surface structures identified from the reflected radiation by means of the data processing device, in particular the identified contaminants.

4. The system as claimed in claim 1, wherein
the mobile terminal is smartglasses, a smartphone, a smart helmet, a tablet, a notebook or a netbook.

5. The system as claimed in claim 4, wherein
the lenses of the smartglasses and/or of the smart helmet comprise a filter, embodied to filter at least 20%, preferably at least 50%, particularly preferably 100%, of light having a wavelength of below 100 nm and having a wavelength of above 490 nm, in particular of above 380 nm.

6. The system as claimed in claim 4, wherein
the lenses of the smartglasses and/or of the smart helmet comprise a filter, embodied to filter at least 20%, preferably at least 50%, particularly preferably 100%, of light having a wavelength in the range of 100 nm to 380 nm.

7. The system as claimed in claim 1, wherein
the at least one radiation source in particular consists of at least one light emitting diode or the latter is comprised by the radiation source.

8. The system as claimed in claim 1, wherein
the at least one radiation source is arranged on the mobile terminal, wherein in particular the at least one radiation source and the at least one optical sensor are movable or are moved equidirectionally

9. The system as claimed in claim 1, wherein
the data processing device conditions the image captured by the at least one sensor by means of imaging methods and identifies specific elements on a surface detected by way of the reflected radiation of the radiation source, in particular contaminants, viruses and/or fungal spores.

10. The system as claimed in claim 9, wherein the data processing device generates a graphical representation of the identified elements and displays it to the user on the display device.

11. The system as claimed in claim 9, wherein
the graphical representation is displayed to the user in a superimposed manner, in particular partly transparently, on the display device of smartglasses and/or a smart helmet.

12. The system as claimed in claim 1, further comprising;
at least one further radiation source is provided in addition to the at least one radiation source on the mobile terminal and additionally emits radiation.

13. The system as claimed in claim 1, further comprising;
at least one further optical sensor is provided in addition to the optical sensor on the mobile terminal, the data of which are evaluated by the data processing device in parallel or alternately with the data of the optical sensor on the mobile terminal.

14. The system as claimed in claim 1, further comprising;
at least one third sensor is comprised in addition to the optical sensor on the mobile terminal in order to detect radiation reflected by the radiation source(s), wherein the data processing is designed and configured to process the data of the at least one third sensor exclusively or in addition to the data of the at least one first sensor or of the at least one further sensor, in order to detect contaminants.

15. The system as claimed in claim 1, wherein
the at least one first sensor and/or the at least one further sensor are/is a camera.

16. The system as claimed in claim 1, wherein
the at least one radiation source and/or the at least one further radiation source emit(s) radiation with a wavelength in a range of 10 nm to 490 nm, in particular in a range of 280 nm to 380 nm, to the extent of, in particular, at least 80% of the emitted radiation energy relative to the total radiation energy of the respective radiation source.

17. The system as claimed in claim 1, wherein
the radiation source is designed and configured to disinfect a surface.

18. A mobile terminal comprising a system as claimed in claim 1.

19. The mobile terminal as claimed in claim 18, wherein the mobile terminal comprises at least the at least one optical sensor, the display device, the at least one radiation source and the data processing device.

20. A method for operating a system or a mobile terminal as claimed in claim 1, comprising the following steps, in particular in this order:
a) activating the radiation source;
b) detecting the radiation of the radiation source reflected by the surroundings by means of the at least one sensor and/or the further sensor; and
c) evaluating the detected sensor data in the data processing device and generating a virtual representation of surface structures, in particular of contaminants.

21. The method as claimed in claim 20, furthermore comprising the following step:
d) displaying the virtual representation of the surface structure on a display device, in particular on smartglasses or a smart helmet, wherein the displaying is displayed in particular in the form of augmented reality as additional information for a user.

22. The method as claimed in claim 20, furthermore comprising the following step, in particular at least at times in parallel with step d:
e) displaying work instructions and/or information in parallel with or as an alternative to displaying the virtual representation of surface structures.

23. The method as claimed in claim 20, furthermore comprising the following step:
f) documenting and recording the changes in the space detected by the at least one sensor or the further sensor and/or the work carried out by the user.

24. The method as claimed in claim 20, furthermore comprising the following step:
g) disinfecting an object and/or a spatial region by means of one of the at least one radiation source or the further radiation source of the system or of the mobile terminal.

25. (canceled)
